(19) United States
(12) Patent Application Publication
(10) Pub. No.: US 2012/0290024 A1
(43) Pub. Date: Nov. 15, 2012

(54) TRANSVENOUS RENAL NERVE MODULATION FOR TREATMENT OF HYPERTENSION, CARDIOVASCULAR DISORDERS, AND CHRONIC RENAL DISEASES

(75) Inventors: Yongxing ZHANG, Irvine, CA (US); Cary HATA, Irvine, CA (US)

(73) Assignee: ST. JUDE MEDICAL, INC., ST. PAUL, MN (US)

(21) Appl. No.: 13/105,419

(22) Filed: May 11, 2011

(51) Int. Cl. A61N 1/365; A61N 1/36

(52) U.S. Cl. 607/3; 607/62; 607/4

(57) ABSTRACT

A transvenous renal nerve modulation system comprises: a blood pressure monitoring device to be implanted in a patient to monitor the patient's blood pressure; one or more transvenous renal nerve modulation leads to be implanted in one or more renal blood vessels of the patient; a pulse generator coupled to the transvenous renal nerve modulation leads to deliver electrical pulses to the one or more transvenous renal nerve modulation leads for modulating renal nerves of the patient; and a control unit coupled to the blood pressure monitoring device and the pulse generator to control delivery of the electrical pulses by the pulse generator based on the patient's blood pressure from the blood pressure monitoring device. The pulse generator delivers high frequency pulses of greater than about 10 Hz to the transvenous renal nerve modulation leads if the patient's blood pressure is greater than a high blood pressure threshold.

### BACKGROUND OF THE INVENTION

The present invention relates generally to nerve modulation and, more specifically, to transvenous renal nerve modulation for the treatment of hypertension, other cardiovascular disorders, and chronic renal diseases.

Hypertension is a major global public health concern. An estimated 30-40% of the adult population in the developed world suffers from this condition. Furthermore, its prevalence is expected to increase, especially in developing countries. Diagnosis and treatment of hypertension remain suboptimal, even in developed countries. Despite the availability of numerous safe and effective pharmacological therapies, including fixed-drug combinations, the percentage of patients achieving adequate blood-pressure control to guideline target values remains low. Much failure of the pharmacological strategy to attain adequate blood-pressure control is attributed to both physician inertia and patient non-compliance and non-adherence to a lifelong pharmacological therapy for a mainly asymptomatic disease. Thus, the development of new approaches for the management of hypertension is a priority. These considerations are especially relevant to patients with so-called resistant hypertension (i.e., those unable to achieve target blood-pressure values despite multiple drug therapies at least three anti-hypertensive agents at their proper doses). Such patients are at high risk of major cardiovascular events.

Hypertension also plays a key role in progressive deterioration of renal function and in the exceedingly high rate of cardiovascular disorders. Clinical and research studies have demonstrated sympathetic nerve activation in not only hypertension but also heart failure, atrial fibrillation, ventricular tachyarrhythmias, long-QT syndrome and other cardiovascular disorders as well as chronic renal diseases.

Renal sympathetic efferent and afferent nerves, which lie within and immediately adjacent to the wall of the renal artery, are crucial for initiation and maintenance of systemic hypertension. Indeed, sympathetic nerve modulation as a therapeutic strategy in hypertension had been considered long before the advent of modern pharmacological therapies. Radical surgical methods for thoracic, abdominal, or pelvic sympathetic denervation had been successful in lowering blood pressure in patients with so-called malignant hypertension. However, these methods were associated with high perioperative morbidity and mortality and long-term complications, including bowel, bladder, and erectile dysfunction, in addition to severe postural hypotension. Renal denervation is the application of a chemical agent, or a surgical procedure, or the application of energy to remove/damage renal nerves to diminish completely the renal nerve functions. This is a complete and permanent block of the renal nerves. Renal denervation diminishes or reduces renal sympathetic nerve activity, increases renal blood flow (RBF), and decreases renal plasma norepinephrine (NE) content. Renal denervation may produce possible complications of thrombosis and renal artery stenosis, and particularly the long-term consequences and effects remain unknown. Furthermore, the renal nerve can regenerate itself, in which case the renal denervation procedure will have to be repeated.

### BRIEF SUMMARY OF THE INVENTION

Embodiments of the present invention are directed to transvenous renal nerve modulation apparatuses and methods for the treatment of hypertension, other cardiovascular disorders, and chronic renal diseases.

In accordance with an aspect of the present invention, a transvenous renal nerve modulation system comprises: a blood pressure monitoring device to be implanted in a patient to monitor the patient's blood pressure; one or more transvenous renal nerve modulation leads to be implanted in one or more renal blood vessels of the patient; a pulse generator coupled to the one or more transvenous renal nerve modulation leads to deliver electrical pulses to the one or more transvenous renal nerve modulation leads for modulating renal nerves of the patient; and a control unit coupled to the blood pressure monitoring device and the pulse generator to control delivery of the electrical pulses by the pulse generator based on the patient's blood pressure from the blood pressure monitoring device. The pulse generator delivers high frequency pulses of greater than about 10 Hz to the one or more transvenous renal nerve modulation leads if the patient's blood pressure is greater than a high blood pressure threshold.

In some embodiments, the patient's blood pressure is greater than the high blood pressure threshold if the patient's systolic blood pressure (SBP) is higher than about 145 mmHg or if the patient's diastolic blood pressure (DBP) is higher than about 90 mmHg, or if difference between the SBP and the DBP is greater than about 55 mmHg. The pulse generator delivers low frequency pulses of less than about 5 Hz to the one or more transvenous renal nerve modulation leads if the patient's blood pressure is less than a low blood pressure threshold. The patient's blood pressure is less than the low blood pressure threshold if the patient's systolic blood pressure (SBP) is lower than about 85 mmHg or if the patient's diastolic blood pressure (DBP) is lower than about 55 mmHg or if difference between the SBP and the DBP is less than about 25 mmHg. The control device and the pulse generator are housed in an implantable module to be implanted in the patient. The blood pressure monitoring device comprises a pressure sensor to be implanted in one of the left atrium of the patient to measure the left atrium pressure, a pulmonary artery of the patient to measure the pulmonary artery pressure, or the left ventricle (LV) of the patient to measure the LV pressure.

In specific embodiments, each transvenous renal nerve modulation lead has one or more modulation electrodes. Each transvenous renal nerve modulation lead has a plurality of modulation electrodes which are selectively energizable by the pulse generator under control of the control unit to transfer modulation energy to the patient. Each transvenous renal nerve modulation lead has one or more sensing electrodes, and the control unit controls operation of the transvenous renal nerve modulation system based on data from the one or more sensing electrodes. A drug source is coupled to the one or more transvenous renal nerve modulation leads to deliver a drug to the one or more renal blood vessels. The control unit controls delivery of the drug from the drug source to the one or more renal blood vessels based on the blood pressure from the blood pressure monitoring device.

In accordance with another aspect of the invention, a transvenous renal nerve modulation method comprises: implanting a blood pressure monitoring device in a patient to monitor the patient's blood pressure; implanting one or more transvenous renal nerve modulation leads in one or more renal blood vessels of the patient; delivering electrical pulses from a pulse generator to the one or more transvenous renal nerve modulation leads for modulating renal nerves of the patient; and controlling delivery of the electrical pulses by the pulse generator to the one or more transvenous renal nerve modulation leads based on the patient's blood pressure from the blood pressure monitoring device. The pulse generator delivers high frequency pulses of greater than about 10 Hz to the one or more transvenous renal nerve modulation leads if the patient's blood pressure is greater than a high blood pressure threshold.

In some embodiments, the patient's blood pressure is greater than the high blood pressure threshold if the patient's systolic blood pressure (SBP) is higher than about 145 mmHg or if the patient's diastolic blood pressure (DBP) is higher than about 90 mmHg, or if difference between the SBP and the DBP is greater than about 55 mmHg. The pulse generator delivers low frequency pulses of less than about 5 Hz to the one or more transvenous renal nerve modulation leads if the patient's blood pressure is less than a low blood pressure threshold. The patient's blood pressure is less than the low blood pressure threshold if the patient's SBP is lower than about 85 mmHg or if the patient's DBP is lower than about 55 mmHg or if difference between the SBP and the DBP is less than about 25 mmHg. The method further comprises implanting the control device and the pulse generator in the patient. Implanting the blood pressure monitoring device comprises implanting a left atrium pressure (LAP) sensor in a left atrium of the patient to measure the left atrium pressure. Implanting the blood pressure monitoring device comprises implanting a pressure sensor in a pulmonary artery of the patient to measure the pulmonary artery pressure. Implanting the blood pressure monitoring device comprises implanting a miniature pressure sensor anchored in the PA for obtaining the patient's left atrial pressure.

In specific embodiments, each transvenous renal nerve modulation lead has a plurality of modulation electrodes, and the method further comprises selectively energizing the plurality of modulation electrodes by the pulse generator to transfer modulation energy to the patient. Each transvenous renal nerve modulation lead has one or more sensing electrodes, and the method further comprises controlling delivery of the electrical pulses by the pulse generator based on data from the one or more sensing electrodes. The method further comprises controlling delivery of a drug to the one or more renal blood vessels based on the blood pressure from the blood pressure monitoring device.

In accordance with another aspect of this invention, a transvenous renal nerve modulation system and cardiac therapy system comprise: a blood pressure monitoring device to be implanted in a patient to monitor the patient's blood pressure; one or more transvenous renal nerve modulation leads to be implanted in one or more renal blood vessels of the patient; a right atrial lead to be implanted in the patient's right atrium for pacing and sensing the right atrium; a coronary venous lead to be implanted in the patient's coronary vein for pacing and sensing the left heart; a pacing or pacing and defibrillation lead implanted in the patient's right ventricle, including the RVOT (RV outflow tract), for pacing and defibrillating the heart; a pulse generator coupled to the one or more transvenous renal nerve modulation leads and cardiac and coronary venous leads to deliver electrical pulses to the one or more transvenous renal nerve modulation leads as well as cardiac leads for modulating renal nerves and providing cardiac resynchronization and other anti-arrhythmia therapy to the patient; and a control unit coupled to the blood pressure monitoring device and the pulse generator to control delivery of the electrical pulses by the pulse generator based on the patient's blood pressure from the blood pressure monitoring device and the patient's conditions as monitored by the right atrial lead, the coronary venous lead, and the pacing or pacing and defibrillation lead to the right ventricle.

These and other features and advantages of the present invention will become apparent to those of ordinary skill in the art in view of the following detailed description of the specific embodiments.

### BRIEF DESCRIPTION OF THE DRAWINGS

FIG. 1 is a block diagram illustrating a system for renal nerve modulation according to an embodiment of the present invention.

FIG. 2 is a schematic illustration of a transvenous renal nerve modulation apparatus utilizing a transseptal left atrial pressure sensor.

FIG. 2A is a schematic illustration of a pressure sensor lead of FIG. 2 which has a terminal pin connected to a standalone device that communicates wirelessly with a pulse generator.

FIG. 3A is a schematic illustration of a transvenous renal nerve modulation apparatus utilizing a pulmonary artery lead pressure sensor.

FIG. 3B is a schematic illustration of a transvenous renal nerve modulation and cardiac resynchronization therapy apparatus.

FIG. 4 shows an example of lead or catheter with an S-shaped or sinusoidal anchoring mechanism.

FIG. 5 shows an example of a lead or catheter with a running or serial loop anchoring mechanism.

FIG. 6 shows an example of a lead or catheter with a spiral-shaped anchoring mechanism.

FIG. 7 shows an example of a lead or catheter with stent-like anchoring mechanism.

FIG. 8 shows an example of a lead or catheter having an S-shaped or sinusoidal distal portion anchoring mechanism and an S-shaped or sinusoidal intermediate portion anchoring mechanism.

FIG. 9 shows an example of a lead or catheter having a running or serial loop distal portion anchoring mechanism and an S-shaped or sinusoidal intermediate portion anchoring mechanism.

FIG. 10 shows an example of a lead or catheter having a distal portion anchoring mechanism for anchoring the lead in the renal vein and a intermediate portion anchoring mechanism for anchoring the lead in the IVC.

FIG. 11 shows an example of a lead or catheter having a drug delivery passageway or channel and one or more fluid elution holes.

FIG. 12 shows a renal lead delivery sheath which has two distal openings for two guidewires to be directed to the right renal vein and the left renal vein.

FIG. 13 shows a renal lead delivery sheath which has a distal opening and a side opening disposed proximally from the distal opening.

### DETAILED DESCRIPTION OF THE INVENTION

In the following detailed description of the invention, reference is made to the accompanying drawings which form a part of the disclosure, and in which are shown by way of illustration, and not of limitation, exemplary embodiments by which the invention may be practiced. In the drawings, like numerals describe substantially similar components throughout the several views. Further, it should be noted that while the detailed description provides various exemplary embodiments, as described below and as illustrated in the drawings, the present invention is not limited to the embodiments described and illustrated herein, but can extend to other embodiments, as would be known or as would become known to those skilled in the art. Reference in the specification to “one embodiment,” “this embodiment,” or “these embodiments” means that a particular feature, structure, or characteristic described in connection with the embodiment is included in at least one embodiment of the invention, and the appearances of these phrases in various places in the specification are not necessarily all referring to the same embodiment. Additionally, in the following detailed description, numerous specific details are set forth in order to provide a thorough understanding of the present invention. However, it will be apparent to one of ordinary skill in the art that these specific details may not all be needed to practice the present invention. In other circumstances, well-known structures, materials, circuits, processes and interfaces have not been described in detail, and/or may be illustrated in block diagram form, so as to not unnecessarily obscure the present invention.

In the following description, relative orientation and placement terminology, such as the terms horizontal, vertical, left, right, top and bottom, is used. It will be appreciated that these terms refer to relative directions and placement in a two dimensional layout with respect to a given orientation of the layout. For a different orientation of the layout, different relative orientation and placement terms may be used to describe the same objects or operations.

Exemplary embodiments of the invention, as will be described in greater detail below, provide transvenous renal nerve modulation apparatuses and methods for the treatment of hypertension.

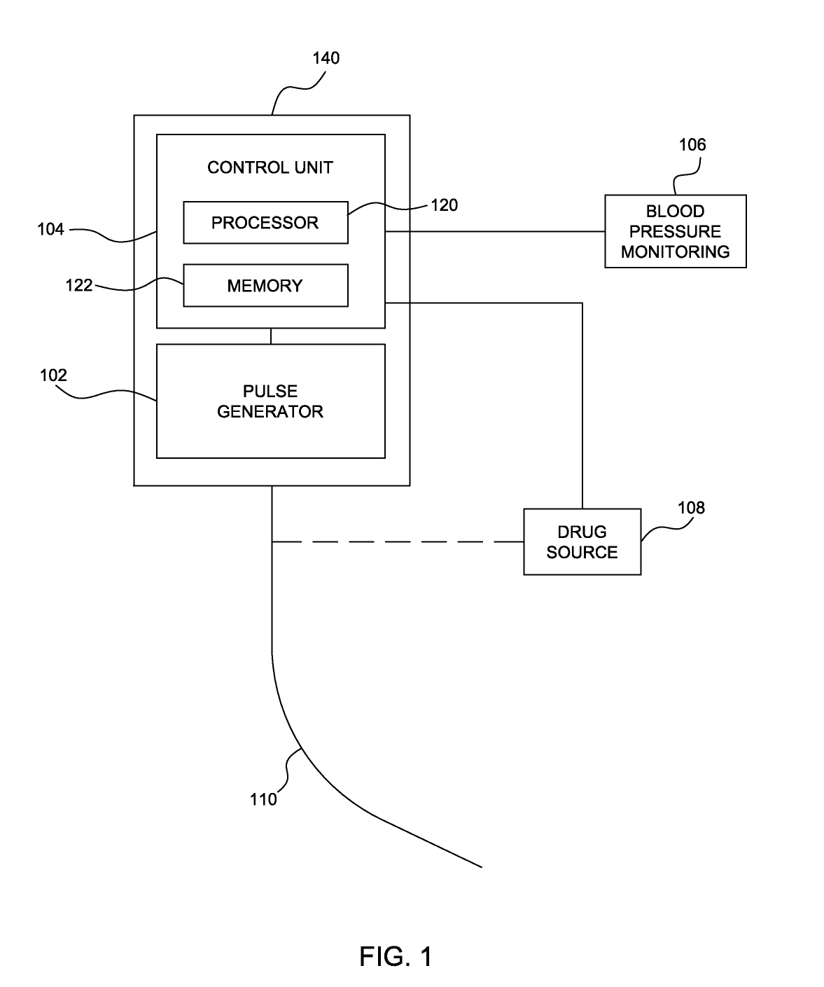

FIG. 1 is a block diagram illustrating a system for renal nerve modulation according to an embodiment of the present invention. The transvenous renal nerve modulation system includes a pulse generator 102, a control unit 104, a blood pressure monitoring device 106, optionally a drug source 108 (e.g., renal nerve blocking agent), and one or more transvenous renal nerve modulation leads 110 that are implanted in the heart and renal veins for hypertension treatment.

The pulse generator 102 delivers energy to the renal nerves via the one or more modulation leads to achieve a therapeutic effect. The therapies involve renal nerve modulation, which is stimulation or inhibition. The pulse generator can be battery powered or rechargeable, or can operate on other types of energy. The pulse generator may deliver a variety of waveforms at different energy/voltage levels and frequencies to provide unipolar, bipolar, and/or multi-polar modulation. Each modulation lead of leads 110 may have its own pulse generator, or a single pulse generator may be used to supply energy to all the modulation leads. The communication of the sensing electrodes and sensors on the leads can occur with wire connection or wireless. The leads 110 can be implanted in the renal vessels, the heart, and other locations in the cardiovascular system. For multi-polar electrodes in a lead electrode configuration, an electrode reposition feature allows the clinician to select which electrode(s) to use for nerve modulation. The modulation electrodes may be selectively energizable to transfer modulation energy to the patient.

Renal nerve stimulation (RNS) is the application of electrical stimuli of low frequency (usually <5 Hz) to activate the renal nerve. RNS activates renal sympathetic nerve activity, increases renal plasma NE content, and decreases renal blood flow (RBF). RNS increases the release of norepinephrine and decreases RBF. Renal nerve electrical inhibition is the application of electrical current with high frequency (usually >10 Hz) or appropriate to inhibit (block) the renal nerves. The renal nerve inhibition is a temporary and reversible renal nerve block, or a temporary and reversible renal denervation.

The control device 104 includes a processor 120 and a memory 122 with embedded hardware and software for processing data and executing programs to monitor the patient (e.g., blood pressure) and perform therapy (e.g., renal nerve modulation, drug therapy). Monitoring data can be collected from the blood pressure monitoring device 106 and/or sensors provided on the lead(s) 110 implanted in the patient. Therapies can be performed by activating the pulse generator 102 to apply energy to modulation electrodes on the lead(s) 110 and/or delivering a drug such as a blocking agent from the drug source 108 through the lead(s) 110 to the patient. A drug delivery mechanism in the form of a miniature or MEMS pump may be used to deliver the drug under the control of the control device 104 (at the desired time, duration, dosage, etc.). Both the electrical modulation and drug delivery can be conducted based on monitoring physiological conditions of the patient, including, for example, blood pressure sensed in the blood pressure monitoring device 106 and sensors on the lead(s) 110. The electrical modulation and drug delivery are conducted based on monitoring physiological conditions in vivo. The control device 104 may include one or more telemetry features for wireless communication with sensor(s) and device(s) that are implanted in the body of the patient or disposed external of the body for patient monitoring, therapeutic purposes, or the like. In a specific embodiment, the control unit 104 and the pulse generator 102 (and optionally the drug source 108) are provided in a single implantable device 140.

The blood pressure monitoring device 106 measures the patient's blood pressure, for example, using a pressure sensor implanted in the patient's heart, i.e., left atrium, pulmonary artery, left ventricle, a coronary blood vessel, or the like.

The therapeutic parameters are programmed into the control unit 104 with the patient's blood pressure and other clinical characteristics. The control unit 104 can be programmed telemetrically. Based on the patient's blood pressure and other cardiovascular data obtained by the blood pressure and cardiovascular monitoring device 106, the pulse generator 102 delivers therapies according to the change of blood pressure. For example, the therapies involve renal nerve modulation which is stimulation or inhibition. The control unit 104 provides closed loop control of the pulse generator 102 for renal nerve modulation. The acceptable blood pressure (BP) has a range between a low BP threshold and a high BP threshold. For example, the systolic blood pressure (SBP) has a range between a low SBP threshold of 85 mmHg and a high SBP threshold of 145 mmHg; the diastolic blood pressure (DBP) has a range between a low DBP threshold of 55 mmHg and a high SBP threshold of 90 mmHg; the difference of SBP and DBP low threshold of 25 mmHg, and a SBP to DBP high difference of 55 mmHg. If the blood pressure is lower than the low BP threshold, the pulse generator 102 applies low frequency pulses (typically less than about 5 Hz) to stimulate or activate the renal sympathetic nerve. If the blood pressure is higher than the high BP threshold, the pulse generator 102 applies high frequency pulses (typically greater than or much greater than about 10 Hz) to inhibit the renal nerve.

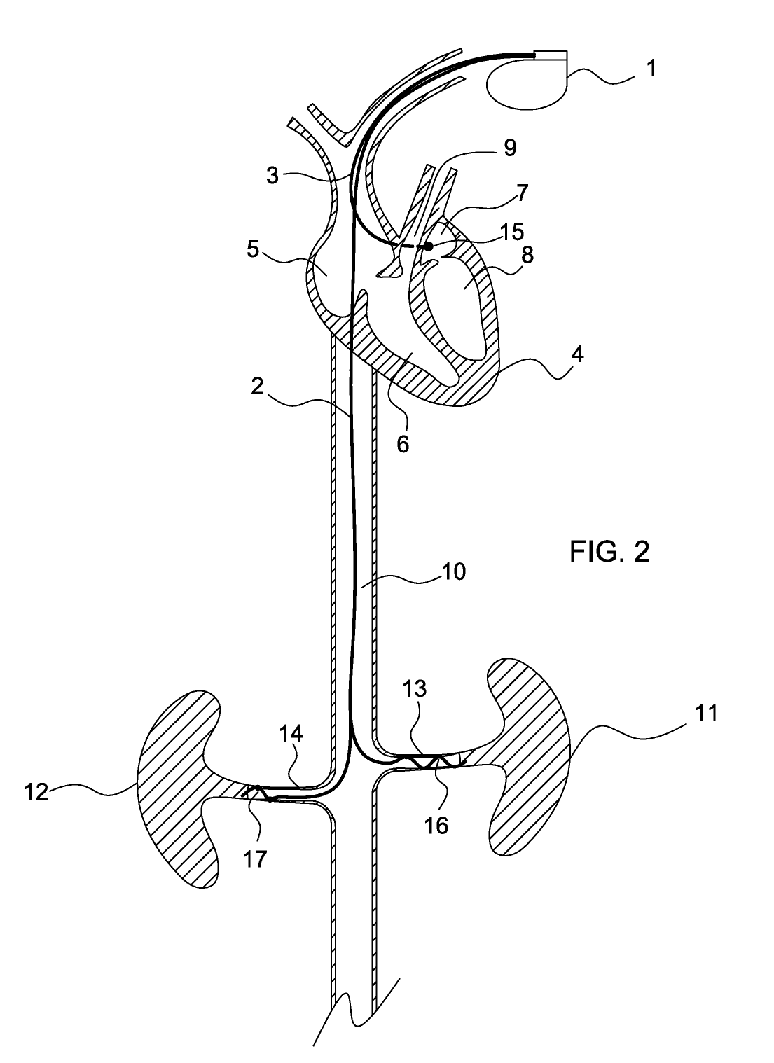

FIG. 2 is a schematic illustration of a transvenous renal nerve modulation apparatus utilizing a transseptal left atrial pressure (LAP) sensor. The apparatus includes an implantable pulse generator 1 coupled to one or two renal nerve modulation leads 2 which are implanted in the patient to modulate the renal nerves. One lead 2 is implanted in either the left renal vein 13 or the right renal vein 14. If two leads 2 are provided, they are implanted respectively in the left and right renal veins. The pulse generator 1 is coupled to a pressure sensor on lead 3 which is implanted transseptally into the left atrium 7 with a pressure sensor 15 for monitoring the left atrial pressure. FIG. 2 shows the right atrium (RA) 5, the right ventricle (RV) 6, the left atrium (LA) 7, the left ventricle (LV) 8, and the RV outflow tract (RVOT) and pulmonary artery (PA) 9 of the heart 4. The renal nerve modulation leads 2 extend through the inferior vena cava (IVC) into the left renal vein 13 on the side of the left kidney 11 and the right renal vein 14 on the side of the right kidney 12. Electrodes 16 are provided on the renal nerve modulation lead 2 in the left renal vein 13. Electrodes 17 are provided on the renal nerve modulation lead 2 in the right renal vein 14. It is noted that the above discussion, as well as the entire description of the present invention, should also cover the application and implantation of the modulation system in the renal arteries.

The LAP sensor 15 is used to monitor the end diastole filling pressure for real time cardiac performance. The LAP sensor 15 can be implanted percutaneously via the femoral or the subclavian vein into the RA 5 and transseptally into the LA 7. It may be fixed in position by one or more folding Nitinol septal fixation anchors or the like. The distal end of the pressure sensor lead 3 is connected to the LAP sensor 15, and the proximal end of the pressure sensor lead 3 has a terminal pin connected to the pulse generator 1 or to a standalone device 20 that communicates wirelessly with the pulse generator 1, as seen in FIG. 2A.

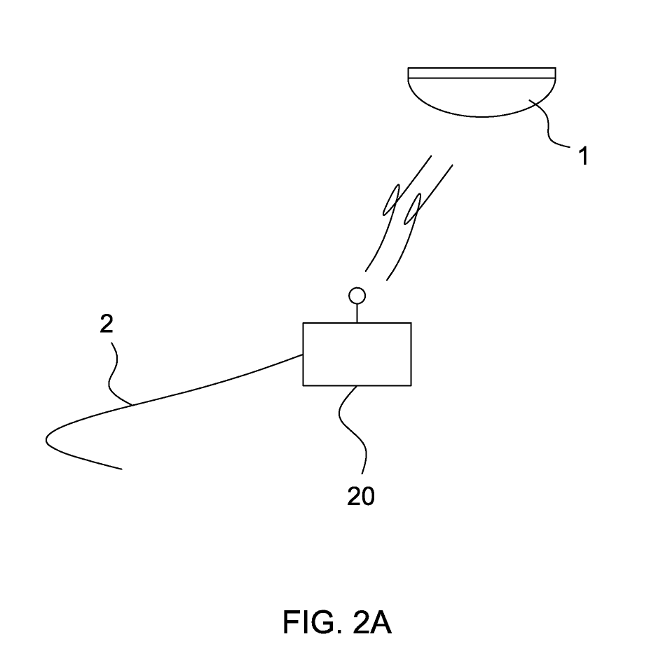

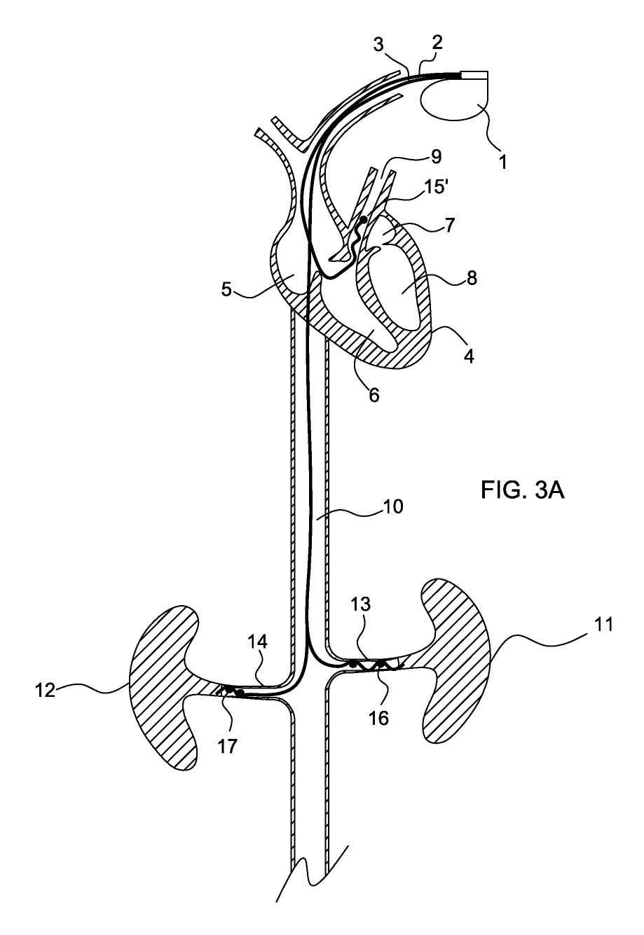

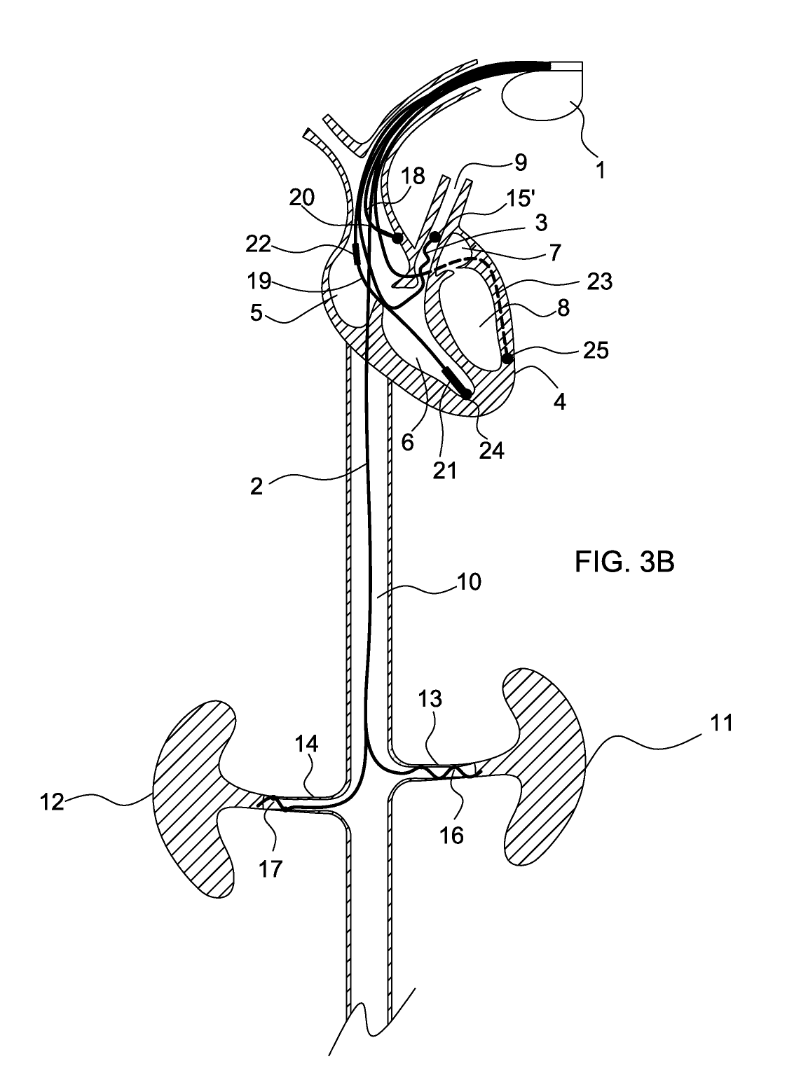

FIG. 3A is a schematic illustration of a transvenous renal nerve modulation apparatus utilizing a pulmonary artery lead pressure sensor. The modulation apparatus of FIG. 3A is generally the same as the modulation apparatus of FIG. 2 except for the following. Instead of a pressure sensor lead 3 which is implanted transseptally into the left atrium 7, FIG. 3A shows a pressure sensor lead 3 which is implanted in the PA 9 for measurement of the wedged pressure via a pressure sensor 15′, which clinically approximate the LA pressure. The PA pressure sensor 15′ can be implanted percutaneously via the femoral or the subclavian vein passing the RVFT into the pulmonary artery 9. It can be mounted in a stent-like structure, or with some other fixation mechanism, implanted in the pulmonary artery 9.

The pressure sensor instrumented on a lead as shown in FIG. 3A may be replaced by a CardioMEMS-type miniature pressure sensor. The CardioMEMS pressure sensor is a miniature pressure sensor having the size of a small paper pin (i.e., it can be as small as about 0.5 mm×2 mm×1.5 mm in size), which is made using the MEMS technology. It can be implanted in the same manner as the PA pressure sensor into the PA. The CardioMEMS pressure sensor can also be a wireless sensor. The fixation mechanism can be an opened hoop exerting a pressure against the PA, or the sensor can be mounted on a stent-like component which is pressed against the PA inner wall with or without an anchoring mechanism such as that of a transcatheter valve anchoring mechanism. CardioMEMS pressure sensors are developed by CardioMEMS in Atlanta, Ga.

The renal leads are for renal nerve modulation (inhibition and stimulation) of the renal sympathetic nerves. The modulation leads can be configured to unipolar, bipolar, or multi-polar modulation. Each renal lead has a terminal connector at the proximal end which is connected to the pulse generator. The distal segment of each modulation lead is preformed for fixation in the renal vein and to achieve good electrode-tissue contact. Because the renal blood vessels (veins and arteries) are subject to displacement during respiration, each lead includes a passive or an active fixation mechanism for fixation in the renal blood vessel. The renal leads can utilize a variety of fixation mechanisms, different conductor designs, and different cross-sectional configurations. The lead has one or more modulation electrodes and may have one or more sensing electrodes. The modulation electrodes can be made of platinum-iridium (PtIr) or some other suitable electrode materials. Examples of sensing electrodes include sensors for sensing temperature, oxygen in blood, catheter tip force or pressure, blood pressure, blood flow, nerve activity, and impedance contact with the renal vein near the modulation electrode.

The lead has an elongated body which extends along a longitudinal axis and which includes a proximal end and a distal portion having a distal end. The preformed shape of the distal portion is for fixation of the lead in the renal vein to prevent dislodgment and better electrode contact with the renal vein. The preformed shape can be two-dimensional (i.e., planar) or three-dimensional, and can be S-shaped, spiral-shaped, etc. The anchoring mechanism may be movable between a collapsed position (for easy delivery) and an expanded position, and anchors the distal portion to the biological cavity such as a renal vein in the expanded position.

FIG. 3B is a schematic illustration of a transvenous renal nerve modulation and cardiac resynchronization therapy apparatus. The renal nerve modulation and cardiac therapy apparatus of FIG. 3B includes an implantable pulse generator 1, a pair of renal nervous modulation leads 2 implanted in the left and right renal veins (or arteries) 13 and 14, and a lead 3 with a pressure sensor 15′ implanted in the PA or RVOT 9, or a pressure sensor of other types (e.g., the LAP sensor implanted in the LA 7) for blood pressure monitoring, a right atrial pacing and/or sensing lead 18 with RA pacing/sensing electrodes 20, a coronary venous lead 23 with electrode 25 implanted in the coronary vein and great cardiac vein or a coronary venous branch vein for pacing the left heart, and a RV pacing lead or a defibrillation lead 19 implanted in the RV 6. The lead 19 has a RV pacing electrode 24 on the distal tip, a RV shocking electrode 21 and a RA/SVC shocking electrode 22. The pulse generator 1 has the function of a pacemaker and defibrillator as well as a renal nerve modulator, an integrated system in communication with the leads/electrodes and sensors as stated above for providing therapies to treat cardiac arrhythmias, heart failure, hypertension, as well as chronic kidney diseases.

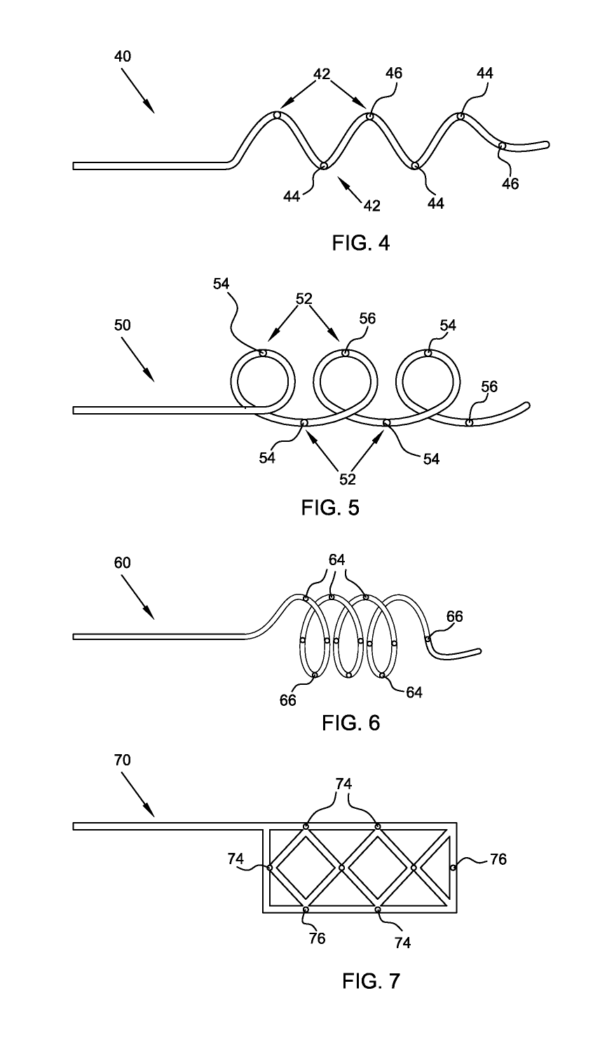

FIG. 4 shows an example of an S-shaped or sinusoidal anchoring mechanism 40. The upper and lower peaks 42 in the lateral direction are configured to contact the tissue wall of the biological cavity of the patient to anchor the lead. A plurality of modulation electrodes 44 are provided at or near the peaks 42. Sensing electrodes 46 typically do not need to contact the tissue wall and hence can be placed at other locations, but some sensing electrodes 46 may be provided at or near the peaks 42 to make tissue contact. FIG. 5 shows an example of a running or serial loop anchoring mechanism 50 with lateral peaks 52, modulation electrodes 54, and sensing electrodes 56. FIG. 6 shows an example of a spiral-shaped anchoring mechanism 60 with modulation electrodes 64 disposed on the exterior of the spiral structure to contact the tissue wall and sensing electrodes 66. This three-dimensional structure has no lateral peaks. FIG. 7 shows an example of a stent-like anchoring mechanism 70 with modulation electrodes 74 disposed on the exterior of the stent structure to contact the tissue wall and sensing electrodes 76. The stent-like structure can be self-expanding or balloon expanded. If a balloon is used, the balloon can be filled with a heated fluid that performs renal denervation by heating the renal nerve. One or more temperature sensors can be provided to monitor the temperature to be used to control the heated denervation. The stent-like structure can be implanted into the renal vein using techniques for implanting stents delivered by a catheter or the like. A renal nerve modulation lead can have one or more stent-like electrodes.

In some embodiments, the lead can be preformed in another region proximal of the distal segment instead of or in addition to the preformed shape in the distal segment. The purpose is to fix the preformed shape portion(s) of the lead to the renal vein near the IVC, while the distal portion of the lead is advanced into a branch of the renal vein to wedge the lead in the renal vein for fixation. The lead may have a bifurcation with two lead branches to be inserted into the left renal vein and the right renal vein, respectively. The renal leads are typically inserted into the renal veins, but it is possible to implant the renal leads in the renal arteries (the other type of blood vessels).

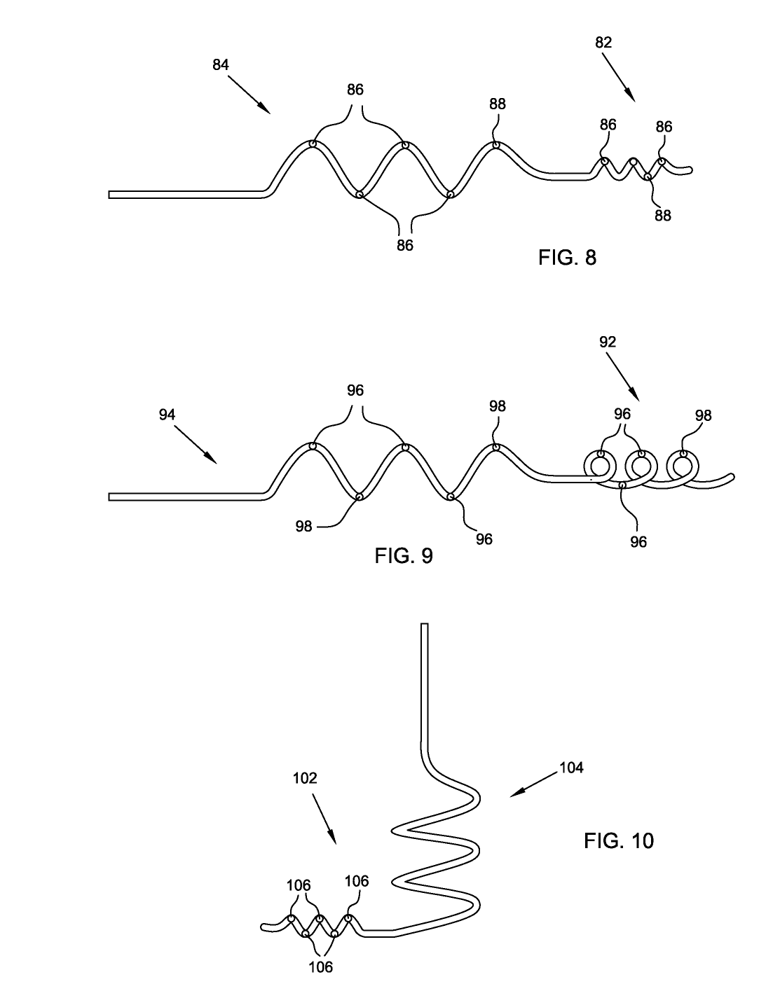

In the embodiments shown in FIGS. 8 and 9, an intermediate portion is disposed between the proximal end and the distal portion. The distal portion includes a distal portion anchoring mechanism to anchor the distal portion to a first biological cavity of a patient. The intermediate portion includes an intermediate portion anchoring mechanism to anchor the intermediate portion to a second biological cavity of the patient. The intermediate portion anchoring mechanism is larger in lateral dimension than the distal portion anchoring mechanism. The lateral dimension is a dimension perpendicular to the longitudinal axis; it may be a lateral length or a lateral area perpendicular to the longitudinal axis. The intermediate portion anchoring mechanism is spaced from the distal portion anchoring mechanism. The second biological cavity is larger than the first biological cavity in the lateral dimension. For example, a maximum lateral dimension of the intermediate portion anchoring mechanism is at least twice as large as a maximum lateral dimension of the distal portion anchoring mechanism. The first biological cavity and the second biological cavity may be in the renal vein and the first biological cavity is closer to the kidney than the second biological cavity. At least one of the distal portion or the intermediate portion includes a plurality of modulation electrodes. At least one of the distal portion anchoring mechanism and the intermediate portion anchoring mechanism is configured to position the modulation electrodes to contact tissue of the patient at multiple locations.

FIG. 8 shows an example of a lead having an S-shaped or sinusoidal distal portion anchoring mechanism 82 and an S-shaped or sinusoidal intermediate portion anchoring mechanism 84, with modulation electrodes 86 and sensing electrodes 88. FIG. 9 shows an example of a lead having a running or serial loop distal portion anchoring mechanism 92 and an S-shaped or sinusoidal intermediate portion anchoring mechanism 94, with modulation electrodes 96 and sensing electrodes 98. The distal portion anchoring mechanism and the intermediate portion anchoring mechanism may have different configurations in addition to being different in size in the lateral dimension.

FIG. 10 shows an example of a lead having a distal portion anchoring mechanism 102 for anchoring the lead in the renal vein and a intermediate portion anchoring mechanism 104 for anchoring the lead in the IVC. Modulation electrodes 106 are provided on the distal portion for renal nerve modulation.

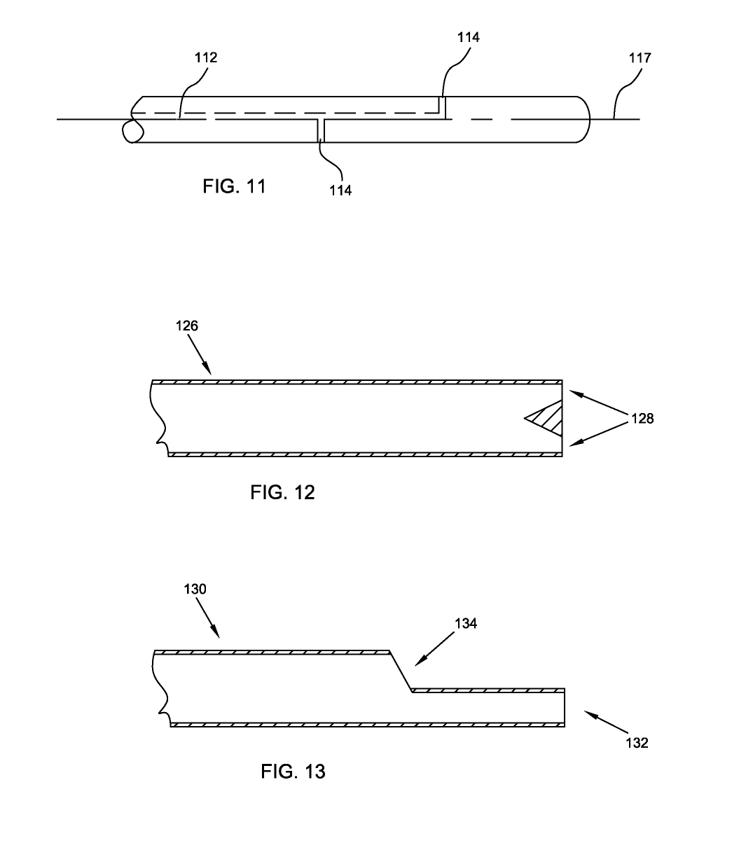

The lead can also include an additional feature of a drug delivery passageway or channel to provide renal nerve or sympathetic blocking drug delivery for renal denervation (or inhibition) using a pharmaceutical agent. The sympathetic blocking agent may include bupivacaine or similar anesthetic agent. The lead may include distal end opening(s), side opening(s), polymeric coated drugs, or the like for elution of the drug into the renal vein. The openings on the lead can also be used for the purpose of cooling the electrode in high-frequency modulation or RF energy delivery. If the lead is an over-the-wire implantable lead with a lumen, the lumen can be used as the drug delivery channel after implantation. Alternatively, a different lumen can be used for drug delivery. FIG. 11 shows an example of a lead having a drug delivery passageway or channel 112 and one or more fluid elution holes 114 coupled to the fluid channel 112. The lead has a longitudinal axis 117 between the proximal end and the distal end. The holes 114 may be disposed on the distal portion and/or the intermediate portion in the embodiments of FIGS. 8 and 9.

The renal lead has an insulation tubing wrapped around a coil conductor. The preformed shape can be achieved by preforming the coil conductor, the insulation tubing, or both. The lead body insulation may be made of high performance medical silicone rubber, polyurethane tubing, or other biocompatible, flexible materials. The conductor can be of multi-fila coil of MP35N-tantalum core wire, platinum clad tantalum core coated with ETFE fluoropolymer (ethylene-tetra-fluoro-ethlene), or some other conductor materials. The conductor can be in the form of coil conductor and/or cable with any suitable cross-sectional design (e.g., co-axial coils, co-radial coils, web etc.). The inner lumen and the external body of the lead may be coated with a coating agent for the purposes of anti-coagulation, anti-thrombosis, anti-infection, and lubrication.

Implantation of the renal lead can be done by stylet delivery, over-the-wire delivery, catheter delivery, or the like. For over-the-wire delivery, the lead has an open lumen from the proximal end to the distal end. If a catheter is used for renal lead delivery, two renal leads can be implanted into the left renal vein and the right renal vein, respectively, using a single catheter insertion. FIG. 12 shows a renal lead delivery catheter 126 which has two distal openings 128 for two guidewires to be directed to the right renal vein and the left renal vein, respectively. FIG. 13 shows a renal lead delivery catheter 130 which has a distal opening 132 for a guide wire to be directed to a renal vein and a side opening 134 disposed proximally from the distal opening 132 to place a slidable secondary lead to reach a renal venous branch.

Similar to pacemaker leads, the renal venous leads can be implanted via the SVC-RA-IVC (superior vena cava to right atrium to inferior vena cava) into the renal veins. One of the advantages of the transvenous renal nerve modulation is the ease and simplicity of the implantation procedure, which can be performed under local anesthesia and takes only about 30 minutes. The following is an example of renal lead implantation procedure.

Step 1: Perform venous access of the lead entry spot in the same way as that for a pacemaker implantation. The lead entry point can be at the subclavian vein, the cephalic vein, the auxiliary vein, or the femoral vein. The vein entry can be accessed by a percutaneous needle insertion or a cut-down.

Step 2: Upon locating the vein entry point, remove the syringe and insert a wire into the vein entry and advance the wire to the SVC.

Step 3: Remove the needle and insert an introducer over the wire, and then remove the wire.

Step 4: Insert a guidewire via the SVC-RA-IVC path to the renal vein and/or introduce a guiding catheter via the SVC-RA-IVC path to the renal vein, remove the introducer.

Step 5: Advance the renal nerve modulation lead over the guidewire into the renal vein. For catheter delivery, remove the guidewire and advance the lead together with an implantation catheter into a targeted renal blood vessel. For stylet delivery, advance a lead, with a stylet inserted therein, into the desired renal vessel location.

Step 6: Partially withdraw the guidewire into the IVC and check to see if the lead stays at the desired location in the renal vein (for OTW or over-the-wire). For catheter delivery, withdraw the implantation catheter. For stylet delivery, withdraw the stylet.

Step 7: Apply modulation to the lead while adjusting the electrode location and electrode modulation until the appropriate modulation is achieved in adjusting the blood pressure.

Step 8: Remove the guidewire from the patient (for OTW). Remove the implantation catheter (for catheter delivery). Remove the stylet, for stylet delivery.

Step 9: Test and make sure the implanted renal nerve modulation apparatus works as desired.

Step 10: Implant the pulse generator by inserting the terminal contact of the lead into the header of the pulse generator and implanting the pulse generator subcutaneously, for instance, in the pectoral region of the patient.

Transvenous renal nerve modulation has many advantages. The implantation procedure is easy and the treatment is simple. The patient is expected to recover quickly after the implantation procedure. The implantation can be performed by electrophysiologists and cardiologists without extensive training. The renal nerve modulation is provided to the patient when and only when it is needed (namely, when the blood pressure is high). There is much less risk of thrombosis and coagulation in the venous system. As compared with other interventional methods, the present method can be readily acceptable by a large number of patients suffering from hypertension. There is significant cost-saving as compared to drug therapy. It should benefit patients for whom drug therapy is not an effective treatment.

In the description, numerous details are set forth for purposes of explanation in order to provide a thorough understanding of the present invention. However, it will be apparent to one skilled in the art that not all of these specific details are required in order to practice the present invention. Additionally, while specific embodiments have been illustrated and described in this specification, those of ordinary skill in the art appreciate that any arrangement that is calculated to achieve the same purpose may be substituted for the specific embodiments disclosed. This disclosure is intended to cover any and all adaptations or variations of the present invention, and it is to be understood that the terms used in the following claims should not be construed to limit the invention to the specific embodiments disclosed in the specification. Rather, the scope of the invention is to be determined entirely by the following claims, which are to be construed in accordance with the established doctrines of claim interpretation, along with the full range of equivalents to which such claims are entitled.

What is claimed is:

1. A transvenous renal nerve modulation system, comprising:
a blood pressure monitoring device to be implanted in a patient to monitor the patient's blood pressure;
one or more transvenous renal nerve modulation leads to be implanted in one or more renal blood vessels of the patient;
a pulse generator coupled to the one or more transvenous renal nerve modulation leads to deliver electrical pulses to the one or more transvenous renal nerve modulation leads for modulating renal nerves of the patient; and
a control unit coupled to the blood pressure monitoring device and the pulse generator to control delivery of the electrical pulses by the pulse generator based on the patient's blood pressure from the blood pressure monitoring device;
wherein the pulse generator delivers high frequency pulses of greater than about 10 Hz to the one or more transvenous renal nerve modulation leads if the patient's blood pressure is greater than a high blood pressure threshold.

2. The system of claim 1,
wherein the patient's blood pressure is greater than the high blood pressure threshold if the patient's systolic blood pressure (SBP) is higher than about 145 mmHg or if the patient's diastolic blood pressure (DBP) is higher than about 90 mmHg, or if difference between the SBP and the DBP is greater than about 55 mmHg.

3. The system of claim 1,
wherein the pulse generator delivers low frequency pulses of less than about 5 Hz to the one or more transvenous renal nerve modulation leads if the patient's blood pressure is less than a low blood pressure threshold.

4. The system of claim 3,
wherein the patient's blood pressure is less than the low blood pressure threshold if the patient's systolic blood pressure (SBP) is lower than about 85 mmHg or if the patient's diastolic blood pressure (DBP) is lower than about 55 mmHg or if difference between the SBP and the DBP is less than about 25 mmHg.

5. The system of claim 1,
wherein the control device and the pulse generator are housed in an implantable module to be implanted in the patient.

6. The system of claim 1,
wherein the blood pressure monitoring device comprises a pressure sensor to be implanted in one of the left atrium of the patient to measure the left atrium pressure, a pulmonary artery of the patient to measure the pulmonary artery pressure, or the left ventricle (LV) of the patient to measure the LV pressure.

7. The system of claim 1,
wherein each transvenous renal nerve modulation lead has one or more modulation electrodes.

8. The system of claim 1,
wherein each transvenous renal nerve modulation lead has a plurality of modulation electrodes which are selectively energizable by the pulse generator under control of the control unit to transfer modulation energy to the patient.

9. The system of claim 1,
wherein each transvenous renal nerve modulation lead has one or more sensing electrodes; and
wherein the control unit controls operation of the transvenous renal nerve modulation system based on data from the one or more sensing electrodes.

10. The system of claim 1, further comprising:
a drug source coupled to the one or more transvenous renal nerve modulation leads to deliver a drug to the one or more renal blood vessels.

11. The system of claim 1,
wherein the control unit controls delivery of the drug from the drug source to the one or more renal blood vessels based on the blood pressure from the blood pressure monitoring device.

12. A transvenous renal nerve modulation method, comprising:
implanting a blood pressure monitoring device in a patient to monitor the patient's blood pressure;
implanting one or more transvenous renal nerve modulation leads in one or more renal blood vessels of the patient;
delivering electrical pulses from a pulse generator to the one or more transvenous renal nerve modulation leads for modulating renal nerves of the patient; and
controlling delivery of the electrical pulses by the pulse generator to the one or more transvenous renal nerve modulation leads based on the patient's blood pressure from the blood pressure monitoring device;
wherein the pulse generator delivers high frequency pulses of greater than about 10 Hz to the one or more transvenous renal nerve modulation leads if the patient's blood pressure is greater than a high blood pressure threshold.

13. The method of claim 12,
wherein the patient's blood pressure is greater than the high blood pressure threshold if the patient's systolic blood pressure (SBP) is higher than about 145 mmHg or if the patient's diastolic blood pressure (DBP) is higher than about 90 mmHg, or if difference between the SBP and the DBP is greater than about 55 mmHg.

14. The method of claim 12,
wherein the pulse generator delivers low frequency pulses of less than about 5 Hz to the one or more transvenous renal nerve modulation leads if the patient's blood pressure is less than a low blood pressure threshold.

15. The method of claim 14,
wherein the patient's blood pressure is less than the low blood pressure threshold if the patient's systolic blood pressure (SBP) is lower than about 85 mmHg or if the patient's diastolic blood pressure (DBP) is lower than about 55 mmHg or if difference between the SBP and the DBP is less than about 25 mmHg.

16. The method of claim 12, further comprising:
implanting the control device and the pulse generator in the patient.

17. The method of claim 12,
wherein implanting the blood pressure monitoring device comprises implanting a left atrium pressure (LAP) sensor in a left atrium of the patient to measure the left atrium pressure.

18. The method of claim 12,
wherein implanting the blood pressure monitoring device comprises implanting a pressure sensor in a pulmonary artery of the patient to measure the pulmonary artery pressure.

19. The method of claim 12,
wherein implanting the blood pressure monitoring device comprises implanting a miniature pressure sensor anchored in the PA for obtaining the patient's left atrial pressure.

20. The method of claim 12, wherein each transvenous renal nerve modulation lead has a plurality of modulation electrodes, the method further comprising:
selectively energizing the plurality of modulation electrodes by the pulse generator to transfer modulation energy to the patient.

21. The method of claim 12, wherein each transvenous renal nerve modulation lead has one or more sensing electrodes, the method further comprising:
controlling delivery of the electrical pulses by the pulse generator based on data from the one or more sensing electrodes.

22. The method of claim 12, further comprising:
controlling delivery of a drug to the one or more renal blood vessels based on the blood pressure from the blood pressure monitoring device.

23. A transvenous renal nerve modulation system and cardiac therapy system, comprising:
a blood pressure monitoring device to be implanted in a patient to monitor the patient's blood pressure;
one or more transvenous renal nerve modulation leads to be implanted in one or more renal blood vessels of the patient;
a right atrial lead to be implanted in the patient's right atrium for pacing and sensing the right atrium;
a coronary venous lead to be implanted in the patient's coronary vein for pacing and sensing the left heart;
a pacing or pacing and defibrillation lead implanted in the patient's right ventricle, including the RVOT (RV outflow tract), for pacing and defibrillating the heart;
a pulse generator coupled to the one or more transvenous renal nerve modulation leads and cardiac and coronary venous leads to deliver electrical pulses to the one or more transvenous renal nerve modulation leads as well as cardiac leads for modulating renal nerves and providing cardiac resynchronization and other anti-arrhythmia therapy to the patient; and
a control unit coupled to the blood pressure monitoring device and the pulse generator to control delivery of the electrical pulses by the pulse generator based on the patient's blood pressure from the blood pressure monitoring device and the patient's conditions as monitored by the right atrial lead, the coronary venous lead, and the pacing or pacing and defibrillation lead to the right ventricle.
